(19) United States
(12) Patent Application Publication
(10) Pub. No.: US 2022/0230755 A1
(43) Pub. Date: Jul. 21, 2022

(54) Systems and Methods for Cognitive Diagnostics for Neurological Disorders: Parkinson's Disease and Comorbid Depression

(71) Applicants: Rutgers, The State University of New Jersey, New Brunswick, NJ (US); Al-Quds University, Palestine (PS)

(72) Inventor: Mohammad Herzallah, Newark, NJ (US)

(73) Assignees: Rutgers, The State University of New Jersey, New Brunswick, NJ (US); Al-Quds University, Palestine (PS)

(21) Appl. No.: 17/716,464

(22) Filed: Apr. 8, 2022

Related U.S. Application Data
(63) Continuation of application No. PCT/US20/54796, filed on Oct. 8, 2020.
Application No. PCT/US20/54796 is a continuation-in-part of application No. 17/055,709, filed on Nov. 16, 2020.
(60) Provisional application No. 62/912,593, filed on Oct. 8, 2019.
(60) Provisional application No. 62/672,726, filed on May 17, 2018.

(51) Int. Cl. G16H 50/20; A61B 5/00; A61B 5/16

(52) U.S. Cl. CPC G16H 50/20; A61B 5/7264; A61B 5/7275; A61B 5/165; A61B 5/7475; A61B 5/742; A61B 5/4082; A61B 5/162

(57) ABSTRACT

A system for diagnosing a neurological disorder, Parkinson's Disease, and a comorbid mental health condition, major depressive disorder is provided. The system comprises a smart device and a device including a memory and a processor. The smart device allows a participant to perform a cognitive task and the device receives data collected from the smart device in connection with the cognitive task performed by the participant. The device determines whether the participant has Parkinson's Disease based on the data collected and via a classification algorithm. If the participant has Parkinson's Disease, the device determines whether the participant has comorbid major depressive disorder.

### CROSS-REFERENCE TO RELATED APPLICATIONS

This application is a 35 U.S.C. 111(a) continuation application of International Application No. PCT/US2020/054796 filed Oct. 8, 2020, which claims the benefit of U.S. Provisional Patent Application No. 62/912,593 filed on Oct. 8, 2019. This application is also a continuation-in-part of U.S. application Ser. No. 17/055,709 filed on Nov. 16, 2020, which is a U.S. National Phase Application under 35 U.S.C. 371 of International Application No. PCT/US2019/032929 filed May 17, 2019, which claims the benefit of U.S. Provisional Patent Application No. 62/672,726 filed on May 17, 2018. The entire disclosures of these applications are expressly incorporated herein by reference.

### BACKGROUND

### Technical Field

The present disclosure relates generally to the field of cognitive diagnostics. More particularly, the present disclosure relates to systems and methods for cognitive diagnostics in connection with Parkinson's disease, comorbid major depressive disorder and response to antidepressants.

### Related Art

Parkinson's Disease (PD) is a neurological disease that affects specific brain cells and produces symptoms that include muscle rigidity, tremors, and changes in speech and gait. Mental health is extremely important in PD. Although common in other chronic diseases, research suggests that depression and anxiety are even more common in PD. It is estimated that at least 50 percent of those diagnosed with PD will experience some form of comorbid major depressive disorder (“MDD”) during their illness, and up to 40 percent will experience anxiety disorders. Most current solutions for early or initial diagnosis of Parkinson's and comorbid MDD are performed using rating scales or questionnaires with tests performed by healthcare providers when patients report specific symptoms.

MDD is characterized by a long-lasting depressed mood or marked loss of interest or pleasure in all or nearly all activities. Antidepressants, including serotonin-specific reuptake inhibitors (hereinafter “SSRI”), can remediate depressive symptoms in a substantial proportion of patients suffering from MDD. It has been hypothesized that SSRIs achieve their therapeutic effect, in part, by modifying synaptic availability of serotonin and possibly also by enhancing neurogenesis in the hippocampal region. Yet, little is known about the underlying brain structure and neurochemistry in MDD. As a result, MDD diagnosis is based primarily on overt behavioral symptoms. Moreover, such diagnoses are given in a long interview with a medical professional and/or based on a form that is filled out by a patient or caretaker. Despite being accurate, such procedures for diagnosing MDD can take a long time to complete and require regular visits to professionals. Moreover, most patients with MDD do not respond positively to antidepressants and the current procedures for diagnosing MDD do not predict whether a patient will respond to antidepressants at all.

PD and MDD are discussed in the paper entitled “Depression Reduces Accuracy While Parkinsonism Slows Response Time for Processing Positive Feedback in Patients with Parkinson's Disease with Comorbid Major Depressive Disorder Tested on a Probabilistic Category-Learning Task,” by Herzallah, et al., Frontiers in Psychiatry, June 2017, Vol. 8, Art. 84.

### SUMMARY

The present disclosure provides a computer system and method which can collect data from a participant. The participant can interact with a computer device (e.g., a tablet or smartphone) through a short (e.g., ˜10 minutes) feedback-based probabilistic classification cognitive task (hereinafter “FPCT”) during which data can be collected. The data can be processed by the computer device or a remote device in communication with the computer device over a network. The processing of the data can determine attributes of a patient in connection with the dissociation of learning from positive versus negative feedback or other forms of feedback-based learning (e.g., correct feedback versus incorrect feedback or reinforcement learning). The computer device can make this determination based on mathematical models and artificial intelligence approaches to extract additional measures. Based on the output of the computer device, a diagnosis of Parkinson's disease can be made. In addition, the results thereby generated can be used to assess whether the patient also has a comorbid major depressive disorder.

### BRIEF DESCRIPTION OF THE DRAWINGS

The foregoing features of the invention will be apparent from the following Detailed Description, taken in connection with the accompanying drawings, in which:

FIG. 1 is a drawing illustrating an embodiment of a flow diagram of a system of the present disclosure;

FIGS. 2A-B are drawings showing graphs of a result from testing a first example cognitive task of the system of the present disclosure;

FIGS. 3A-B are drawings showing graphs of a result from testing a second example cognitive task of the system of the present disclosure;

FIGS. 4A-D are drawings showing sample screens of a feedback-based classification task in the system of the present disclosure;

FIGS. 5A-H are drawings showing graphs of testing results of the system of the present disclosure;

FIGS. 6A-B are drawings showing two classification graphs for tests conducted in connection with the system of the present disclosure;

FIGS. 7A-C are drawings showing graphs of results of another test performed on the system of the present disclosure;

FIG. 8 is a graph illustrating mean positive and negative bias before and after treatment in connection with a test of the system of the present disclosure;

FIG. 9 is a diagram illustrating hardware and software components of the system of the present disclosure;

FIG. 10 is a diagram illustrating hardware and software components of a computer system on which the system of the present disclosure could be implemented;

FIG. 11 is a drawing illustrating another aspect of a flow diagram of a system of the present disclosure;

FIG. 12 is a schematic illustration of the system and method of the present disclosure for use in connection with Parkinson's disease;

FIG. 13 is a drawing showing a classification graph for tests conducted in connection with the system of the present disclosure; and

FIG. 14 is a drawing showing a classification graph for tests conducted in connection with the system of the present disclosure.

### DETAILED DESCRIPTION

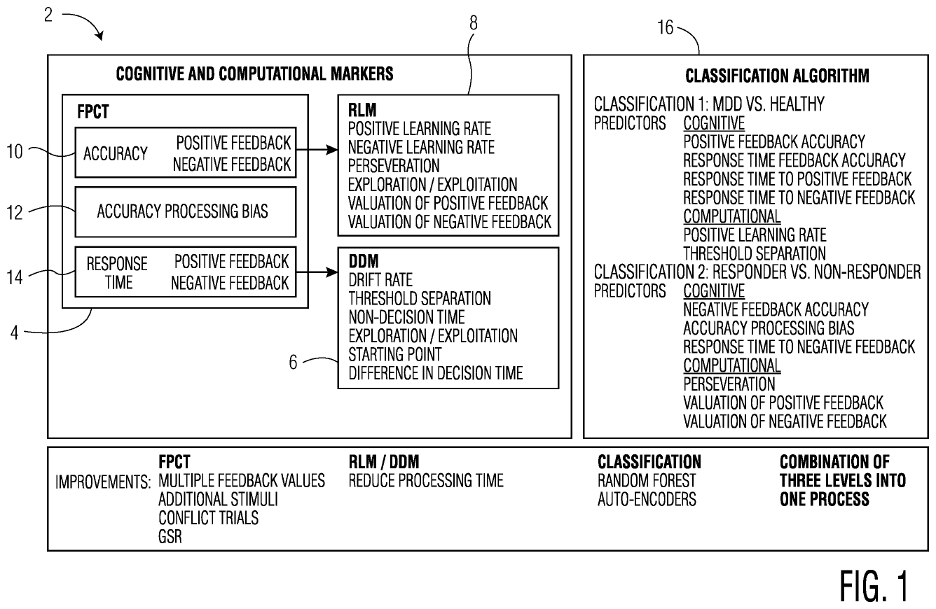

The present disclosure relates to systems and methods for cognitive diagnostics in connection with major depressive disorder and response to antidepressants, as discussed in detail below in connection with FIGS. 1-14.

The present disclosure uses Major Depressive Disorder (“MDD”) as an example of a psychiatric disorder, however, the system of the present disclosure can be used to diagnose any psychiatric disorder, including, but not limited to, post-traumatic stress disorder, obsessive compulsive disorder, schizophrenia, and other anxiety spectrum disorders. Moreover, the present disclosure refers to antidepressants and/or serotonin-specific reuptake inhibitors (hereinafter “SSRI”) as examples of treatment, however, the system of the present disclosure can be used to predict whether a patient will respond to any number of other treatment modalities such as psychotherapy and others.

The present disclosure provides a computer system and method which can collect data from one or more patients. These patients can interact with a computer device (e.g., a tablet or smartphone) through a short (e.g., ˜10 minutes) feedback-based probabilistic classification cognitive task (hereinafter “FPCT”) during which data can be collected. The data can be processed by the computer device or a remote device in communication with the computer device over a network. The computer device can be a local device for a closed-circuit system. The processing of the data can determine attributes of a patient in connection with the dissociation of learning from positive versus negative feedback. The computer device can make this determination based on mathematical models and artificial intelligence approaches to extract additional measures. Based on the output of the computer device, a diagnosis of major depressive disorder (hereinafter “MDD”) can be made. In addition, the results thereby generated can be used to predict whether the patient will respond to antidepressants.

FIG. 1 is a drawing illustrating an embodiment of a flow diagram 2 of the present disclosure. The flow diagram 2 includes cognitive and computational and artificial intelligence markers having a FPCT step 4, variants of the Q-learning reinforcement learning model (RLM) 6, and variants of the drift-diffusion model (DDM) 8. The FPCT step 4 can be for collecting information relating to the results of the FPCT task a patient performed. The FPCT step 4 can include an accuracy component 10, an accuracy processing bias component 12, and a response time component 14. The accuracy component 10 can include factors relating to positive and negative feedback as will be explained in greater detail below. The response time component 14 can also include factors relating to positive and negative feedback as will be discussed in greater detail below.

The FPCT step 4 can output its collected cognitive data such as the accuracy component 10 and the response time component 14 for processing by various computational models and artificial intelligence approaches. In particular, the accuracy component 10 can output its data for processing by the RLM models 8 which can be used to assess parameters related to learning accuracy. Moreover, the response time component 14 can output its data for processing by the DDM models 6 which can be used for assessing parameters related to response time distributions. Cognitive data from the FPCT step 4 and outputs from the DDM computational models 6 and the RLM computational models 8 can be sent to a binomial or multinomial logistic regression model 16 which can accurately determine MDD patients from healthy subjects. Further, the binomial or multinomial logistic regression model 16 can use the same data to accurately determine responders and non-responders to antidepressants. The multinomial logistic regression model 16 can include one or more classification algorithms and artificial intelligence approaches to make these determinations. For example, with respect to diagnosing a patient with MDD, cognitive predictors can include, but are not limited to, learning accuracy from positive feedback, response time to positive feedback, learning accuracy from negative feedback, and response time to negative feedback. With respect to diagnosing a patient with MDD, computational predictors can include, but are not limited to, positive learning rate, negative learning rate, separation threshold, difference in the speed of response for the execution of responses, and drift rate for negative feedback. With respect to determining whether a patient will respond to treatment, cognitive parameters include, but are not limited to, learning accuracy from negative feedback, accuracy processing bias, response time to negative feedback, and response time to positive feedback. With respect to determining whether a patient will respond to treatment, computational parameters include, but are not limited to, preservation, valuation of positive feedback, valuation of negative feedback, separation threshold, and starting point of evidence for decision making.

Examples of cognitive tasks will now be explained in greater detail. This learning task requires participants to learn a sequence of events leading to reward. One example of a cognitive task can be sequence learning and context generalization. It should be noted that the sequence learning and context generalization and chaining tasks are merely examples of a type of task that can be used. The present disclosure is not limited to the exact methodologies of the sequence learning and context generalization tasks described herein. Other variations of the tasks can be used, and the following sequence learning and context generalization task is for illustrative purposes. In the first phase of the task, a computer device can generate a screen which shows a first room (Room 1) with three doors (A1, A2, A3), each identified by its own color. The computer device can allow a participant to choose one of the doors. The computer device can set the correct response as door A1, which can lead to a reward, such as a treasure chest. The incorrect responses can be set as doors A2 or A3, which can lead to a locked door. If the participant selects an incorrect door, the subjects can be prompted to try again. Once the participant learns that door A1 is associated with a reward, the computer device can present the participant with another room (Room 2). This room can have three new colored doors (B1, B2, B3). The computer device can set the incorrect responses to doors B2 and B3 which can lead to a locked door. The computer device can also set the correct response to door B1 which can lead to Room 1, in which the participant would again be presented with the doors A1, A2, and A3 where the same door as previously presented would lead to the reward and the other doors would lead to locked doors. This will allow the participant to learn an association where selecting B1 and then A1 leads to a reward. Once this new association is learned, a new room (Room 3) can be added to the sequence where doors C1, C2, and C3 are presented to a participant. C2 and C3 can be set to lead to a locked door while C1 can lead to Room 2 as discussed above. Now the participant will learn an association where selecting C1, B1, and A1 leads to a reward. Once this association is learned, the participant can be presented with Room 4 with doors D1, D2, and D3. D2 and D3 can be set as incorrect responses and D1 (the correct response) can lead to Room 3. Here, the participant can learn an association that selecting D1, C1, B1, and A1 leads to a reward. It should be noted that the above process is not limited to a three-door situation with a specific number door having the reward. The above cognitive task is merely an example task that can be used in the system of the present disclosure. Nevertheless, the system of the present disclosure can include other cognitive tasks for chaining and sequence mechanisms with context generalization. The above process can be seen in Table 1 below.

TABLE 1
The Sequence Learning with Context Generalization Paradigm

| | | Doors | |
|---|---|---|---|
| Phase | Description | shown | Correct response |
| Practice | Cue-association | P₁P₂P₃ | P₁→reward |
| Sequence- | Chain step A | A₁A₂A₃ | A₁→ reward |
| Learning | Chain step B | B₁B₂B₃ | B₁ →A₁ → reward |
| | Chain step C | C₁C₂C₃ | C₁ →B₁ →A₁ → reward |
| | Chain step D | D₁D₂D₃ | D₁ →C₁ →B₁ →A₁ → reward |
| Context | Example | D₁B₁X₁ | D₁ →C₁ →B₁ →A₁ → reward |
| Generali- | generali- | | |
| zation | zation trial | | |
| Retest | Cue-association | Y₁Y₂Y₃ | Y₁→ reward |

In the context generalization phase as shown above in Table 1, generalization to novel task demands can be tested by presenting various novel incorrect doors as distractors along with a correct door choice in each room. This can require participants to learn the correct response and context associations to obtain the reward as shown in Table 1.

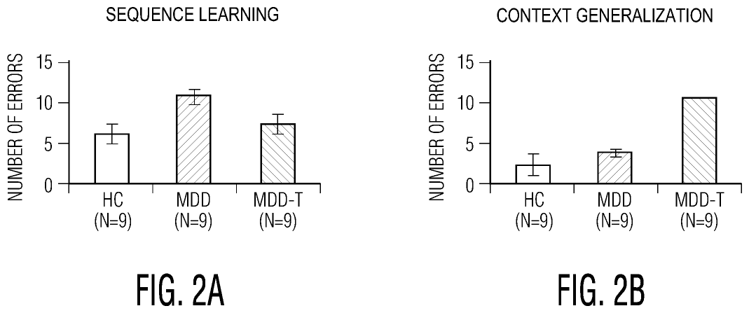

FIGS. 2A-B are graphs which show an example result of testing the above cognitive task. As can be seen, FIG. 2A shows performance on the sequence learning and context generalization task such as the mean number of errors on the sequence-learning phase of the task (chain steps A-D as shown in Table 1). FIG. 2B also shows the mean numbers of errors on the context generalization phase. In the graphs of FIGS. 2A-B, MDD represents medication naïve patients, MDD-T represents patients on medication, and HC represents healthy control subjects. The results show that persons with MDD that are not being treated with medication tend to make many errors on the initial learning/chaining phase, but persons with MDD on medication treatment make many errors in the contextual generalization phase. Univariate ANOVA (alpha=0.05) indicated a significant group difference in the chaining phase results [F(2,24)=4.25, p=0.026, partial n²=0.261] as well as the context generalization phase results [F(2,24)=16.90, p<0.001, partial n²=0.59]. In the sequence-learning phase results, an HSD post hoc test revealed a significant difference between MDD and HCs and between MDD and MDD-T (p<0.05), but not between HCs and MDD-T. In the context generalization phase, an HSD post hoc test revealed a significant difference between MDD-T and HCs, and between MDD-T and MDD (p<0.001), but not between HCs and MDD. An a priori power analysis of one-way ANOVA, done to compute the number of subjects required per group to get a power of 95%, showed that a sample of 48 subjects (16 per group) can be needed to achieve the mentioned power level on the chaining ANOVA, and a total sample of 15 subjects (5 per group) to achieve 95% power level on the context generalization ANOVA.

A second example cognitive task will now be explained in greater detail. The second cognitive task can use a reward-and-punishment-based computer-learning task for weather prediction. In each phase of the task, a computer device can generate four stimuli such as abstract geometric paintings. A participant can view a painting and the device can ask the participant whether that painting predicts rainy weather or sunny weather. The computer device can be programmed so that choosing an answer with respect to two of the stimuli (e.g., paintings) provide feedback for correct answers and incorrect answers result in no feedback. The computer device can also be programmed so that choosing an answer in connection with the other two stimuli provide feedback for incorrect answers and no feedback is given for correct answers. Among both the reward-trained and punishment-trained cues, equal numbers can be associated with rainy weather and sunny weather. All four cues can be intermixed during training. This task is not limited to any specific methodology and can include other tasks related to reward-and-punishment mechanisms.

The cognitive tasks described in the present disclosure can also have the ability to change based on user input providing a dynamic functionality. In particular, the cognitive tasks can change a stimulus or task or question based on a user's prior response(s). For example, if a user is answering questions correctly, the system can increase the difficulty of a subsequent question. Moreover, if a user is answering questions incorrectly, the system can decrease the difficulty of a subsequent question. In this way, the cognitive tasks of the present disclosure are tailored to a user's abilities. Furthermore, the system can change a task to a different task based on the user's input. The system can take into account a plurality of different trials and present a tailored subsequent trial to a user. Accordingly, the systems and methods of the present disclosure can function as a closed loop system for diagnosing mental health conditions and responsiveness to treatments.

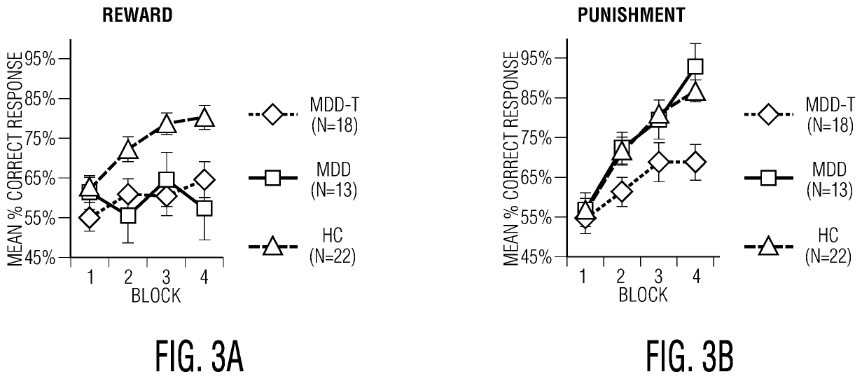

FIGS. 3A-B are graphs which show an example result of testing the above cognitive task. The results tested 13 medication-naïve MDD, 18 MDD-T (Treated, on medication), and 22 healthy controls (HC). FIGS. 3A-B show performance on the two types of trials of the reward and punishment learning task. For example, the mean number of correct responses in the four phases for the reward stimuli is shown in FIG. 3A and the mean number of correct responses in the four phases for the punishment stimuli is shown in FIG. 3B. As noted above, MDD represents patients who are medication naïve and MDD-T represents patients on medication. As can be seen in FIG. 3A, the results show no difference in performance on reward training between MDD and MDD-T being impaired in that phase using one-sample t-test to assess learning higher than chance. In a one-way ANOVA (Bonferroni correction of a=0.025 to protect the level of significance), using the 4th block of reward and punishment trials as the dependent variable, there existed a significant effect of group on learning from punishment [F(2,27)=4.821, p=0.016, n²=0.249] but not on learning from reward [F(2,27)=0.49, p=0.618]. A post hoc analysis of the group effect on 4^{th}-block punishment learning revealed a significant difference between MDD-T patients and MDD patients, and between MDD-T patients and HC (p<0.05). A priori power analysis for ANOVA revealed that the test can have a total of 51 subjects (17 per group) to obtain a power of 95%.

As noted above, the system of the present disclosure can collect data of the participants progress in the above example cognitive tasks and variations thereof. The system of the present disclosure can process this data using a binomial or multinomial logistic regression algorithm to classify subjects as either having MDD or not, and if they do have MDD, whether the subjects would respond to certain medications such as antidepressants. Other classification approaches can be used such as random forest, auto-encoders, or other artificial intelligence and machine learning approaches. Random forest or auto-encoders can offer, in some circumstances, better and quicker results, and can utilize a greater number of predictors. Furthermore, the system of the present disclosure can use the Softmax function in making its classification determinations. It should be noted that the above tasks can be performed in a relatively short period of time (e.g., 15 minutes).

The system of the present disclosure can collect data relating to the time it takes for a participant to respond to the scenarios discussed herein. Depending on the time it takes for the participant to respond, the classification algorithm of the system of the present disclosure can take this information as an input and make determinations regarding MDD and ability to respond to treatments for MDD. As noted above, the data gathered during the cognitive tasks and used by the classification algorithms and artificial intelligence approaches can include, but is not limited to, accuracy of correct answers, incorrect answers, response time, response time as the task progresses, learning progress, and how much the participants value positive and negative feedback. These data points can be processed by the classification algorithm and artificial intelligence approaches to make a determination as to whether a patient has a particular psychiatric disorder and whether that patient will respond to treatment.

The system of the present disclosure can vary the amount of positive/negative feedback associated with stimuli. With learning accuracy in positive and negative feedback being one of the key cognitive predictors, and valuation of feedback being one of the key computational predictors, the system can add new stimuli to the current FPCT with various amounts of positive and negative feedback to get clearer results related to feedback processing.

The system of the present disclosure can also use conflict trials while diagnosing MDD and responsiveness to medications. For example, in some cases, there can be a feedback processing bias that can differentiate clinically depressed vs. non-depressed subjects as well as responders and non-responders. The subject can learn the feedback associated with each stimulus, and it can be expected that subjects develop preferences to stimuli associated with particular feedback. Accordingly, conflict trials can be used to account for these factors.

The system of the present disclosure can also add multiple phases with more stimuli. In particular, the MDD state and potential response to treatment can be expressed cognitively as preferential learning of particular stimuli with particular feedback. Therefore, adding more stimuli while escalating the level of complexity of the FPCT can refine the underlying factors for preferential learning which improves the efficiency of the classification model.

The system of the present disclosure can add galvanic skin response (GSR) or an eye-tracker to assess eye movements as well as pupil size as additional predictors. By adding GSR, eye-tracking, or electroencephalography (EEG), the system can present an unbiased physiological measure to accompany the cognitive measures from the FPCT. Sensors and electrodes can be placed on a patient's body, their eyes, and/or their scalp which can gather physiological data which can be communicated to a computer device in the system of the present disclosure. This computer device can process the data from the sensor to determine the emotions (e.g., happiness, fear, etc.) felt by the patient while completing the tasks described herein. Data from the eye-tracker can also be analyzed to specify the points of focus as well as changes in pupil size. Data from EEG can track changes in brain electrical activity during the FPCT or at baseline (before/after cognitive testing). The classification algorithm can receive these data as input and can use such information in providing enhanced classifications as to a diagnosis and whether a patient will respond to treatment and the best treatment to offer.

The system of the present disclosure can also apply the above processes and cognitive tasks to diagnose other psychiatric disorders including, but not limited to, post-traumatic stress disorder, obsessive compulsive disorder, schizophrenia, and other anxiety spectrum disorders.

The system of the present disclosure can also test patients after they have received antidepressants to determine whether they responded to the treatment or whether they are still depressed. This can be done by leveraging the cognitive tasks discussed above.

The system can also predict a patient's response to psychotherapy in addition to antidepressants. The classification algorithm and artificial intelligence approaches as discussed above can use the data captured from the tasks and make a determination as to whether a patient will respond to psychotherapy. The system can also determine whether antidepressants or psychotherapy will be better for a given patient based on the cognitive tasks discussed above.

A test with respect to the system of the present disclosure will now be described in greater detail. This test includes 67 medication-naïve patients with MDD and 16 matched healthy controls from various clinics in the Palestine area. A positive and negative feedback classification task for weather prediction was used given by Table 2 below:

TABLE 2

| | Probability | Probability | |
|---|---|---|---|
| Stimulus | category A (%) | category B (%) | Feedback |
| S1 | 90 | 10 | If correct: +25 |
| S2 | 10 | 90 | If incorrect: no feedback |
| S3 | 90 | 10 | If correct: no feedback |
| S4 | 10 | 90 | If incorrect: −25 |

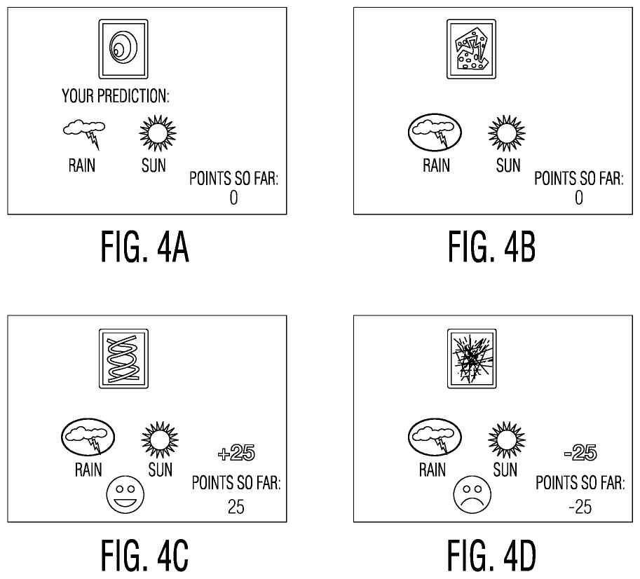

FIGS. 4A-B are drawings showing sample screens of a feedback-based classification task. These are the screens that were used in the above trial. On each trial, a participant saw one of four stimuli and was asked whether this stimulus predicts rainy or sunny weather. In screen 4B, no feedback is given for incorrect answers in positive feedback stimuli or correct answers in negative feedback stimuli. As shown in screen 4C, for positive feedback stimuli, correct responses receive positive feedback with visual feedback and twenty five points of winnings. As shown in screen 4D, for negative feedback, incorrect responses get negative feedback with visual feedback and the loss of 25 points. In the FCPT task, the subject sees one of four stimuli (abstract geometric paintings) and is asked to make a prediction. For example, the subject is asked whether that stimulus predicts Rain or Sun. Two of the stimuli are trained using only positive feedback for correct answers (incorrect answers result in no feedback). The other two stimuli are trained using only negative feedback for incorrect answers (correct answers result in no feedback). Among both the positive-feedback-trained and negative-feedback-trained cues, one is more strongly associated with Rain and the other with Sun. These associations are probabilistic, so that, for example, a rain-preferred cue is associated with 90% Rain and 10% Sun.

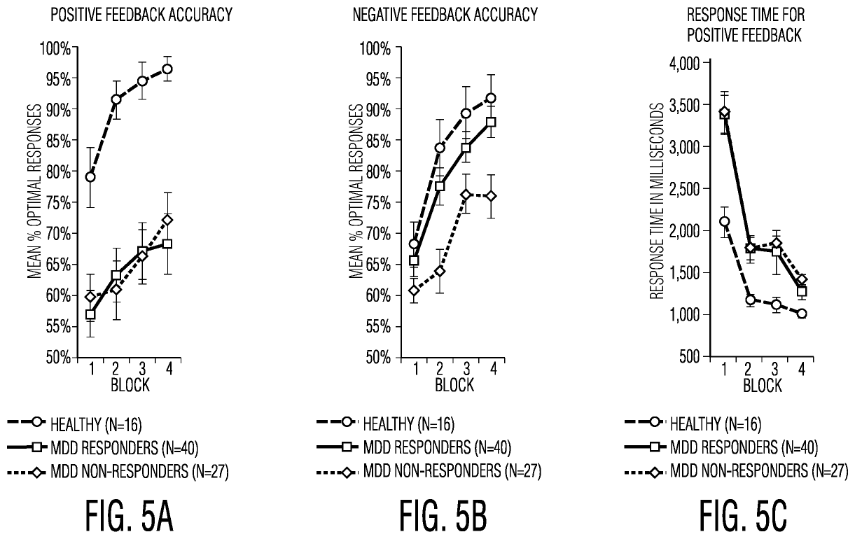

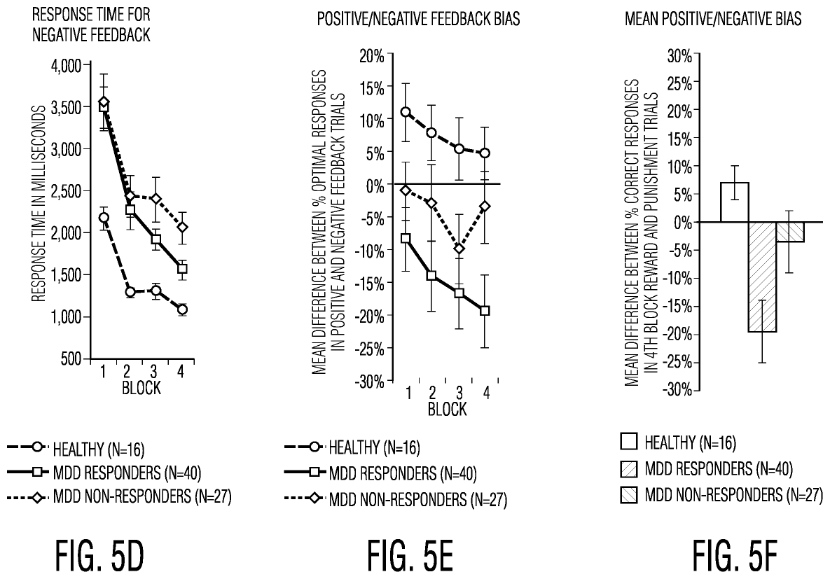

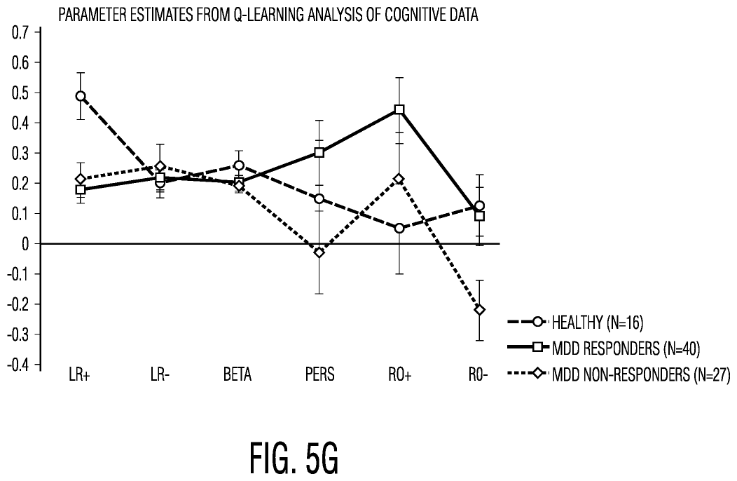

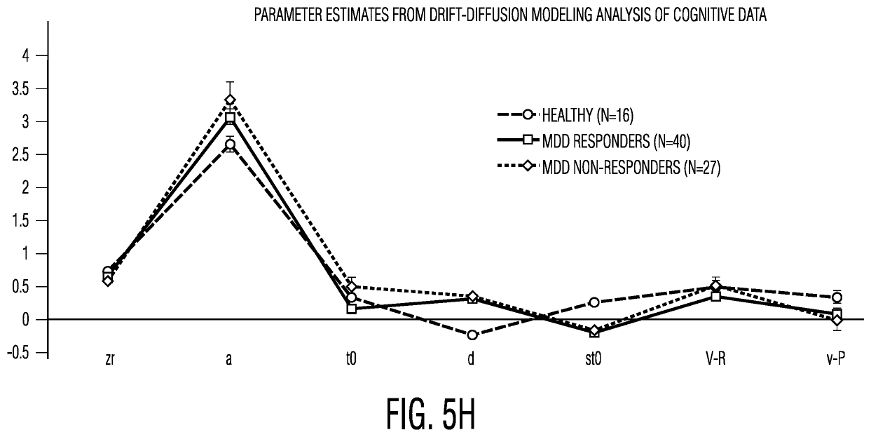

The above test used a variant of the Q-learning trial-by-trial computational analysis to calculate estimates for the following parameters: learning rate with positive prediction error (LR+); learning rate from negative prediction error (LR−); preservation; noise (beta); and valuation of feedback (RO+, RO−). It also used a variant of the DDM trial-by-trial computational analysis to calculate estimates for the following parameters: drift rate (v) for positive-feedback and negative-feedback; threshold separation (a); relative starting point (zr); non-decision time (t0); and difference in decision time for correct and incorrect responses (d). The results of the above test can be seen in FIGS. 5A-H. As can be seen in FIGS. 5A and 5B, cognitive and computational analysis results show learning accuracy in positive and negative feedback trials. FIGS. 5C and 5D show response time to positive and negative feedback stimuli. FIGS. 5E and 5F show positive/negative accuracy bias. FIG. 5G shows parameter estimates using a 6-parameter Q-learning model. FIG. 5H shows parameter estimates using a 6-parameter DDM analysis.

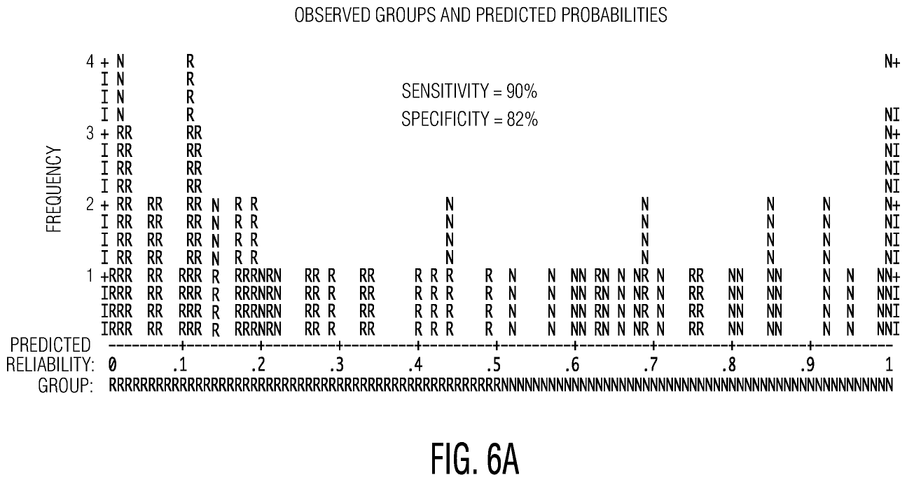

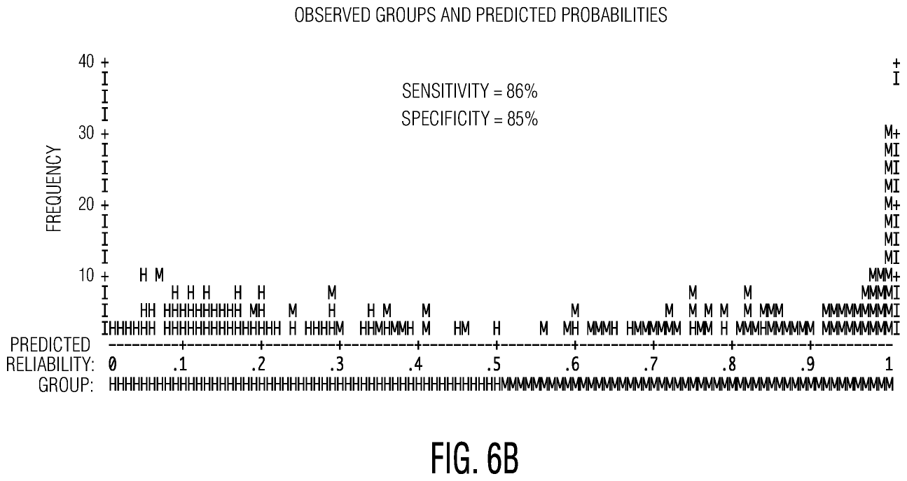

FIGS. 6A-B are drawings showing classification graphs for the above test conducted in connection with the system of the present disclosure. As can be seen, a forward binomial logistic regression classification graph shows a predicted probability of membership for MDD SSRI responder vs. non-responder in FIG. 6A and MDD vs. healthy in FIG. 6B. The cutoff value can be 0.50. In FIG. 6A, R denotes a responder and N denotes a non-responder. In FIG. 6B, M denotes MDD and H denotes healthy subject. Each symbol represents two and a half cases. Four symbols on the graph represent one case.

The above test shows learning accuracy and response time to positive feedback and learning accuracy and response time to negative feedback can differentiate potential patients with MDD from healthy subjects. It also shows learning accuracy and response time to negative feedback can a priori differentiate potential SSRI-responders and non-responders at the medication-naïve level. These results provide an easy to use diagnostic tool that can have immediate clinical relevance. Moreover, it shows lower positive learning rate and learning noise in patients with MDD than healthy subjects. SSRI non-responders exhibit higher levels of preservation during learning. Further, SSRI non-responders value no-feedback in negative feedback trials as negative, which can explain the deficit in negative feedback learning accuracy. It also shows higher threshold separation (a), higher difference in decision time for correct and incorrect responses (d), lower non-decision time (st0), and lower drift rate for negative feedback (v-p). This could explain the slower response time in patients with MDD. In addition, MDD is associated with a selective deficit in learning from positive feedback. SSRI non-responders have balanced learning from positive and negative feedback at the medication-naïve state similar to healthy subjects.

Another test with respect to a positive and negative feedback probabilistic classification task was conducted in connection with the system of the present disclosure. In particular, 67 medication naïve patients with MDD and 16 matched healthy controls participated in Palestine. Patients with MDD were retested 4-6 weeks after starting paroxetine regimen. Healthy controls were also retested at a similar time interval. Response to paroxetine was considered positive if a patient's Beck Depression Inventory II score dropped 50 percent from baseline, and the patient screened negative for MDD on the Mini International Neuropsychiatric Interview. The same positive and negative feedback probabilistic feedback classification task for weather prediction can be used with a feedback structure given by Table 2 above. A similar user interface can also be used as shown in FIGS. 4A-D.

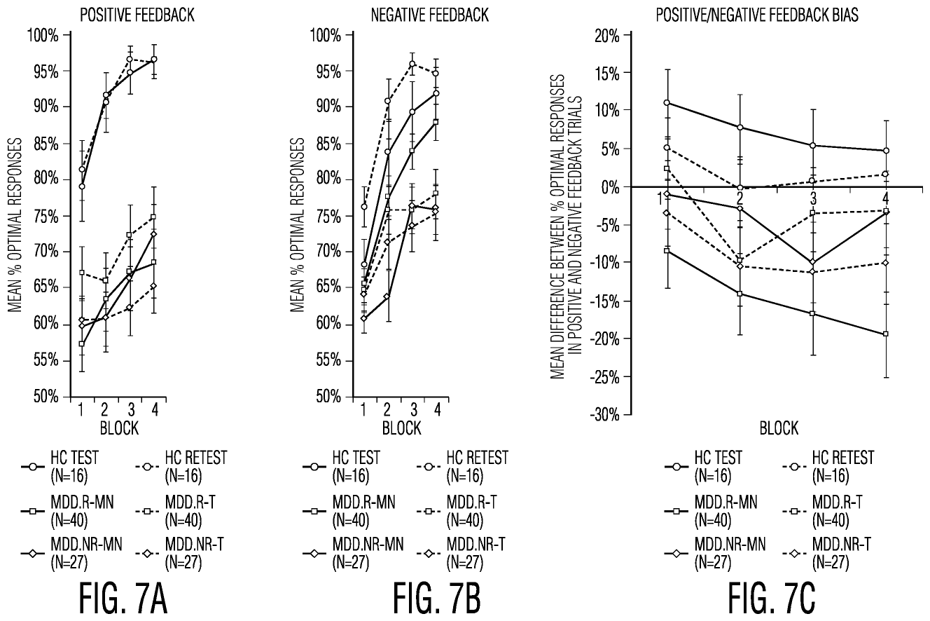

FIGS. 7A-C shows results of the test discussed above. Performance on the positive and negative feedback learning task is shown. In FIG. 7A, the graph shows that the mean number of optimal responses in the four phases for the positive feedback stimuli. In FIG. 7B, the graph shows the mean number of optimal responses in the four phases for the negative feedback stimuli. In FIG. 7C, the graph shows the mean difference between percentage optimal responses in positive and negative feedback trials per block. MDD.R-MN represents participants that are medication-naïve with MDD and who are SSRI responders. MDD.R-T represents participants who are SSRI responders. MDD.NR-MN represents participants who are medication-naïve with MDD who are SSRI non-responders. MDD.NR-T represents participants who are SSRI non-responders. HC test represents healthy controls at baseline and HC retest are healthy controls after 4-6 weeks.

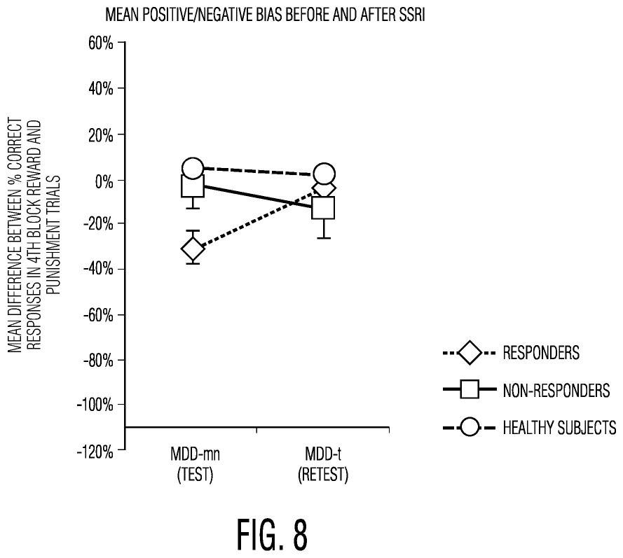

FIG. 8 is a graph illustrating mean positive and negative bias before and after SSRI treatment. As can be seen, FIG. 8 shows a mean difference between percentage optimal responses in positive and negative feedback trials across blocks per testing session before and after SSRI treatment for MDD and at-test and retest for healthy subjects. MDD-mn (test) is medication-naïve baseline for MDD and baseline for healthy subjects. MDD-t (retest) is SSRI-treated retesting for MDD patients 4-6 weeks after SSRI administration and retesting at 4-6 weeks for healthy subjects. The conclusions from this test shows that learning from negative feedback can differentiate potential SSRI-responders and non-responders at the medication-naïve level. Moreover, SSRI-responsive MDD is associated with a selective deficit in learning from positive feedback. Further, SSRI non-responders have balanced learning from positive and negative feedback at the medication-naïve state. Finally, SSRI administration suppresses learning from negative feedback in responders only, thereby bringing positive and negative feedback learning into balance.

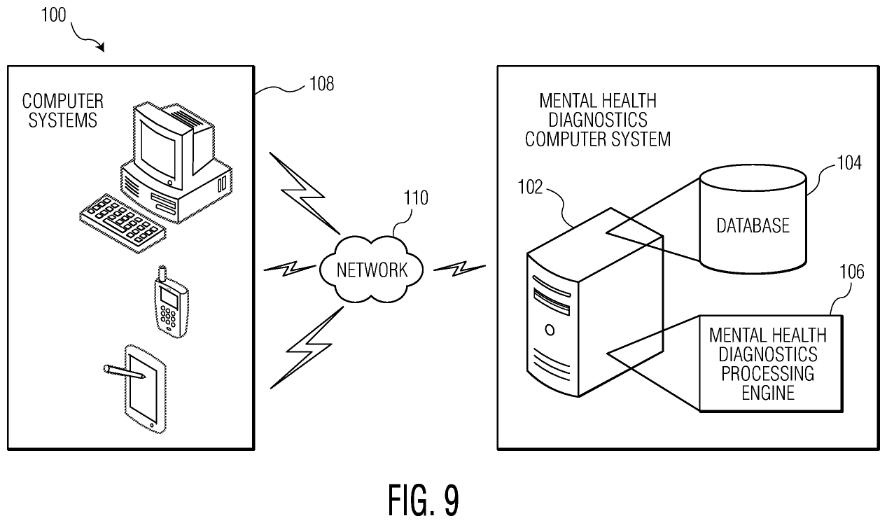

FIG. 9 is diagram illustrating hardware and software components of the system of the present disclosure. A system 100 can include a mental health diagnostics computer system 102. The mental health diagnostics computer system can include a database 104 and a mental health diagnostics processing engine 106. The system 100 can also include a computer system(s) 108 for communicating with the mental health diagnostics computer system 102 over a network 110. The computer systems 108 can be computer devices in which the participants perform the tasks as described above. Network communication could be over the Internet using standard TCP/IP communications protocols (e.g., hypertext transfer protocol (HTTP), secure HTTP (HTTPS), file transfer protocol (FTP), electronic data interchange (EDI), etc.), through a private network connection (e.g., wide-area network (WAN) connection, emails, electronic data interchange (EDI) messages, extensible markup language (XML) messages, file transfer protocol (FTP) file transfers, etc.), or any other suitable wired or wireless electronic communications format. The computer system 108 can also be a smartphone, tables, laptop, or other similar device. The computer system 108 could be any suitable computer server (e.g., a server with an INTEL microprocessor, multiple processors, multiple processing cores) running any suitable operating system (e.g., Windows by Microsoft, Linux, etc.). Alternatively, the computer system could be a field-programmable gate array (FPGA) that can run the mathematical models and artificial intelligence approaches simultaneously upon receipt of the cognitive data in a closed-loop system.

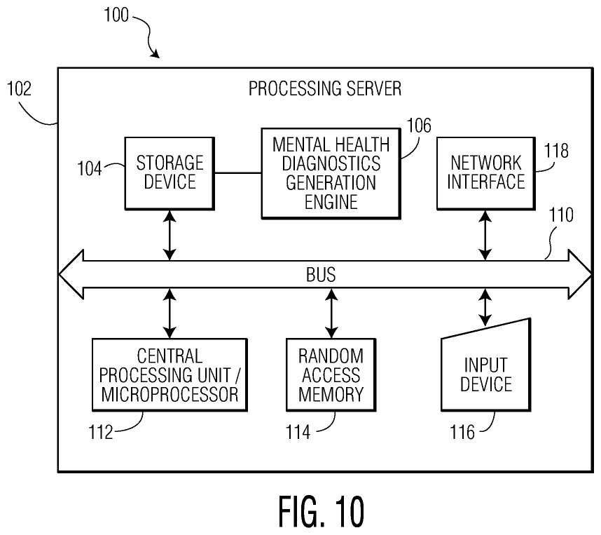

FIG. 10 is a diagram illustrating hardware and software components of a computer system on which the system of the present disclosure could be implemented. The system 100 comprises a processing server 102 which could include a storage device 104, a network interface 118, a communications bus 110, a central processing unit (CPU) (microprocessor) 112, a random access memory (RAM) 114, and one or more input devices 116, such as a keyboard, mouse, etc. The server 102 could also include a display (e.g., liquid crystal display (LCD), cathode ray tube (CRT), etc.). The storage device 104 could comprise any suitable, computer-readable storage medium such as disk, non-volatile memory (e.g., read-only memory (ROM), erasable programmable ROM (EPROM), electrically-erasable programmable ROM (EEPROM), flash memory, field-programmable gate array (FPGA), etc.). The server 102 could be a networked computer system, a personal computer, a smart phone, tablet computer etc. It is noted that the server 102 need not be a networked server, and indeed, could be a stand-alone computer system.

The functionality provided by the present disclosure could be provided by a mental health diagnostics program/engine 106, which could be embodied as computer-readable program code stored on the storage device 104 and executed by the CPU 112 using any suitable, high or low level computing language, such as Python, Java, C, C++, C#, .NET, MATLAB, etc. The network interface 108 could include an Ethernet network interface device, a wireless network interface device, or any other suitable device which permits the server 102 to communicate via the network. The CPU 112 could include any suitable single- or multiple-core microprocessor of any suitable architecture that is capable of implementing and running the mental health diagnostics engine 106 (e.g., Intel processor). The random access memory 114 could include any suitable, high-speed, random access memory typical of most modern computers, such as dynamic RAM (DRAM), etc.

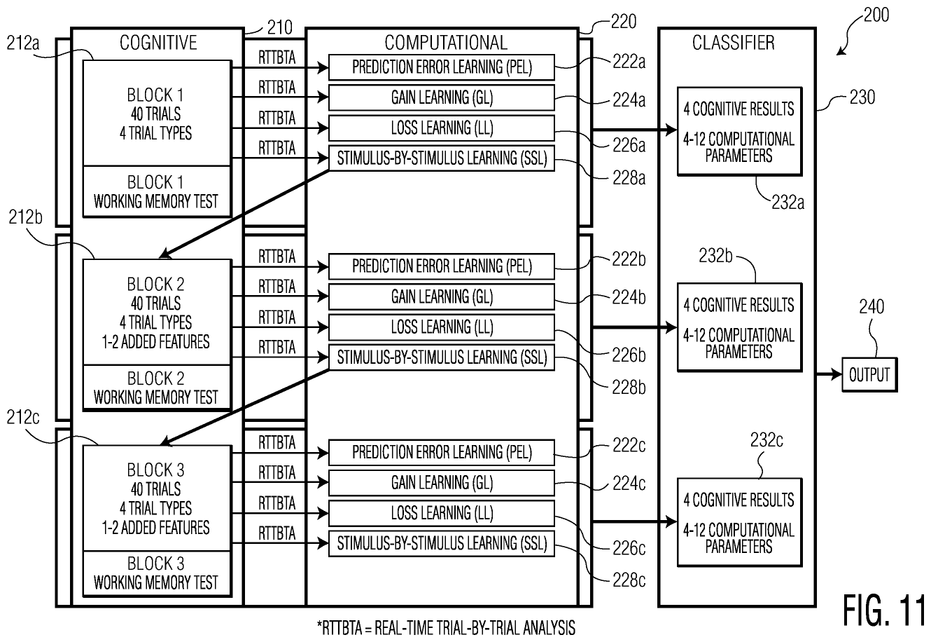

FIG. 11 is a drawing of a flow diagram 200 of another aspect of the system of the present disclosure. The flow diagram 200 illustrates a cognitive component 210, a computational component 220, a classifier component 230 and an output 240. As shown in FIG. 11, an emphasis on the dynamic interaction between the cognitive component 210 and the computational component 220 of the system provides for maximizing an accuracy of the classifier component 230.

The cognitive component 210 includes a plurality of trial blocks 212a, 212b and 212c. Each trial block 212a, 212b, and 212c can include a specified number of trials, a specified number of trial types and a working memory test. Additionally, trial blocks 212b and 212c can include additional features including, but not limited to, outcome reversal, outcome devaluation, gain/loss value modification and delay discounting. These additional features provide for a trial block following a preceding trial block to explore non-dispositive results from the preceding trial block. For example, trial block 212b could be designed with additional features such as gain/loss value modification and delay discounting to explore non-dispositive results from trial block 212a or other cognitive demands related to mental/psychiatric disorders.

The computational component 220 can analyze the cognitive results of each trial block 212a, 212b and 212c utilizing a plurality of modeling and artificial intelligence approaches on a trial by trial basis in real time. Specifically, upon initiation of a cognitive task of a trial block 212a-c, the computational component 220 performs the trial-by-trial computational analysis in real-time while the subject is performing the cognitive task. The plurality of modeling approaches can include, but are not limited to, prediction error learning (PEL) 222a-c, gain learning (GL) 224a-c, loss learning (LL) 226a-c and stimulus-by-stimulus learning (SSL) 228a-c. DDM trial-by-trial analysis of cognitive data can be conducted in parallel. Each of the plurality of modeling approaches can include a set of operating parameters. For example, PEL 222a-c can include operating parameters such as positive learning rate, negative learning rate, and noise, and GL 224a-c can include operating parameters such as gain learning rate, noise, preservation, and valuation of no-feedback. Additionally, LL can include operating parameters such as loss learning rate, noise, preservation, and valuation of no-feedback, and SSL can include operating parameters such as positive learning rate, negative learning rate, noise, preservation and valuation of no-feedback.

Conventional computer-based cognitive tasks suffer from static design that typically does not change throughout an execution of a cognitive task. As such, the system utilizes the cognitive component 210 to design and generate a dynamic cognitive task wherein the performance of the subject influences a design of a subsequent trial block, an addition of various features, and/or the repetition of some of the previously used trial types for further analysis. By fine-tuning a measurement of the cognitive features and the computational parameters, the system can maximize the classification abilities of the classifier component 230.

Specifically, the system utilizes dynamic cognitive task-computational model coupling to maximize the classification abilities of the classifier component 230. For example, for a trial, the cognitive task can transmit a trial type, accuracy, and response time to the various computational models 222a-c, 224a-c, 226a-c and 228a-c to extract parameters of the learning process. Accordingly, over a course of 10-20 trials per trial type, measures of central tendency (e.g., mean and median) as well as variability (e.g., standard deviation, skewness, and kurtosis) can be evaluated and compared to parameters extracted from a large pool of healthy subject data (e.g., a pool of approximately 1000 subjects). Upon ascertaining a difference or a lack of a difference between parameters of the tested subject, the cognitive results and the computational parameters can be adjusted. If the cognitive results and the computational parameters are not adjusted, additional testing of the same type of trials can be resumed in a subsequent trial block. According to the fixed cognitive results and computational parameters, the subsequent trial block can be programmed to test the cognitive dimensions of the subject according to resulting combinations.

The classifier component 230 can execute a plurality of algorithms and artificial intelligence approaches for synthesizing acquired data. For example, the system can implement a multi-layered convolutional neural network (CNN) classifier to emphasize the multi-dimensionality of the dynamic cognitive task-computational model coupling approach and acquired data. Then, according to the cognitive results and computational parameters 232a, 232b and 232c extracted from the subject data, the CNN classifier can assess similarities between results of the subject and pre-defined cognitive/computational patterns that signify respective domains of mental/psychiatric disorders. Subsequently, the system can utilize Random Forest to assign final probabilities.

The present disclosure can be applied to Parkinson's disease and other neurological disorders. It can also be used to diagnose comorbid psychiatric manifestations that affect patients with Parkinson's disease, such as MDD, known as comorbid MDD. Parkinson's disease is diagnosed by the system by varying the amount of positive and/or negative feedback associated with stimuli during feedback-based probabilistic classification cognitive task (FPCT); utilizing reversal trials to potentially implicate the involvement of frontal regions in the disorder; and adding more stimuli while escalating the level of complexity of the FPCT. The system and method of the present disclosure allows a subject play a computer game on a phone/tablet/PC to receive a score for a potential diagnosis with a neurological disorder. This system and method provides an efficient and convenient diagnosis neurological disorders and comorbid mental disorders. This can help the patients and their treating physicians address neurological and mental complaints.

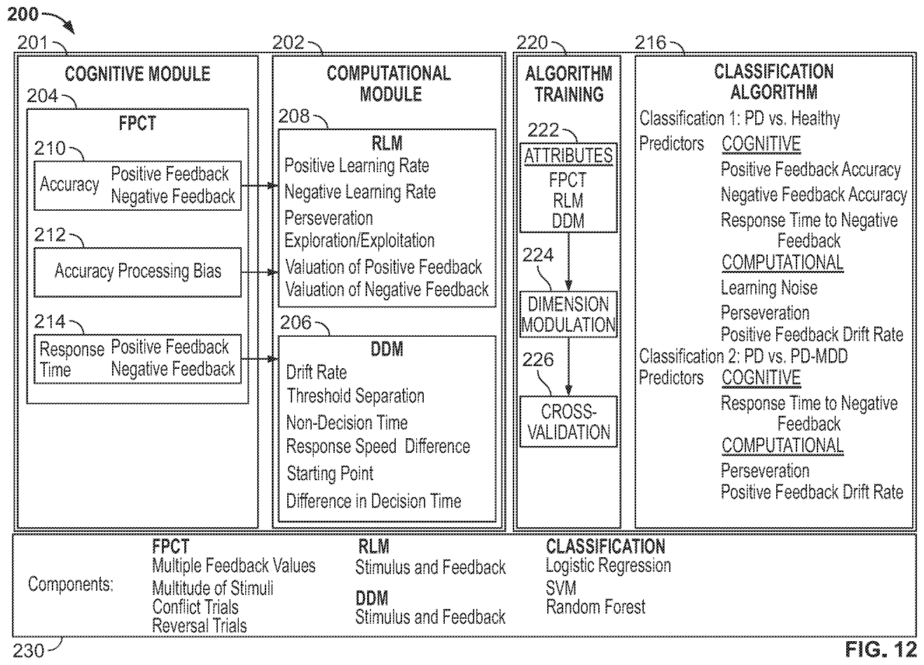

FIG. 12 is a schematic flow illustration of the system and method of the present disclosure for use in connection with Parkinson's disease. As can be seen, the flow of FIG. 12 is similar to the that shown in FIG. 1 and like portions function in a like manner. The flow diagram 200 shows a cognitive module 201 and a computational module 202. The cognitive module 201 shows feedback-based probabilistic classifications (FPCT) 204 including accuracy 210 with positive feedback and negative feedback, accuracy processing bits 212 and response time 214 with positive feedback and negative feedback. The cognitive module 201 is in the form of a cognitive computer task. The computational module 202 includes a reinforcement learning module (RLM) 208 that calculates positive learning rates, negative learning rates, perseveration, exploration/exploitation, valuation of positive feedback and valuation of negative feedback. The drift diffusion module (DDM) 208 calculates drift rate, threshold separation, non-decision time, exploration/exploitation, starting point and difference in decision time. The computational module 202 scales up the data from the cognitive module 201 to generate more dimensions or features.

The classification algorithm 216 distinguishes between subjects with Parkinson's disease and healthy subjects based on cognitive predictors, including positive feedback accuracy, negative feedback accuracy and response time to negative feedback, and based on computational predictors, including learning noise, perseveration and positive feedback drift rate. The classification algorithm also distinguishes between subjects with Parkinson's disease that have and do not have comorbid mental disorder based on cognitive predictors including response time to negative feedback, and based on computational predictors, including perseveration and positive feedback drift rate. An algorithm training component 220 is used to process and store data acquired over time and includes attributes 222 for FPCT, RLM and DDM, dimension modulation 224 such as random tree embedding to determine the separation line of those having Parkinson's disease and those that do not, and cross validation 226 where the process is repeated to increase certainty.

Other components 230 can include, for FPCT, multiple feedback values wherein the reward/punishment values can be modified to accumulate more data for quicker and more efficient diagnosis, multitude of stimuli wherein the computer task can be changed based on the performance of the subject, conflict trials wherein the computer task can be optimized for a subject, and reversal trials which can assess control issues and control of inhibitions. Other components 230 can include RLM stimulus and feedback and DDM stimulus and feedback. Other components can also include classification algorithms, including logistic regression, support vector machine (SVM) which looks for plane of separation of subjects and random forest which includes multiple decision trees.

When a subject performs the computer-based task in the cognitive module 201, five cognitive attributes are generated, including: positive feedback accuracy, negative feedback accuracy, feedback bias accuracy, positive feedback response time, and negative feedback response time. These cognitive attributes are analyzed by RLM and DDM computational models in the computational module 202 to produce 12 computational attributes. In particular, RLM models 208 produce the following attributes: positive learning rate, negative learning rate, perseveration, noise, and valuation of feedback. DDM models 206 produce the following attributes: drift rate, threshold separation, non-decision time, difference in decision time, response speed difference, and starting point. The cognitive and computational results from the training dataset are then used to train a classification algorithm, such as logistic regression, support-vector machines, decision trees, or random forest. Training confirms the attributes that will contribute to the most efficient classification process. Cross-validation approaches are then used to confirm that the trained model can sufficiently classify all of the assigned categories and can be generalized to new data with similar properties. The trained algorithm is then used as the classification algorithm on new data from new subjects.

Using data collected from a short (˜10 minutes) FPCT that allows for the dissociation of learning from positive versus negative feedback alongside mathematical models to extract additional measures, PD can be diagnosed and an assessment can be made about whether patients have comorbid clinical depression (PD-MDD). The collected cognitive data (accuracy of choices and response time) are processed using two computational models: (1) A Q-learning RLM to assess parameters related to learning accuracy, and (2) A DDM to assess parameters related to response time distributions. Cognitive data from the FPCT and parameters from the two computational models are then fed into a multinomial logistic regression model that can differentiate PD patients from healthy subjects in virtually all of the cases. Further, these results can differentiate PD patients with PDD in virtually all of the cases. If there is a determination that the subject has PD and/or PD-MDD, the subject can be further evaluated and/or provided with medical treatment.

The parameters that differentiate healthy and PD subjects include:

COGNITIVELY (1) Learning accuracy from positive feedback, (2) Learning accuracy from negative feedback, and (3) Response time to negative feedback;

COMPUTATIONALLY: (1) Learning noise, (2) Perseveration, (3) Positive feedback drift-rate, and (4) Non-decision time parameters.

The parameters that differentiate PD patients with PDD include:

COGNITIVELY: (1) Response time to negative feedback;

COMPUTATIONALLY: (1) Learning noise, (2) Positive feedback drift-rate parameters.

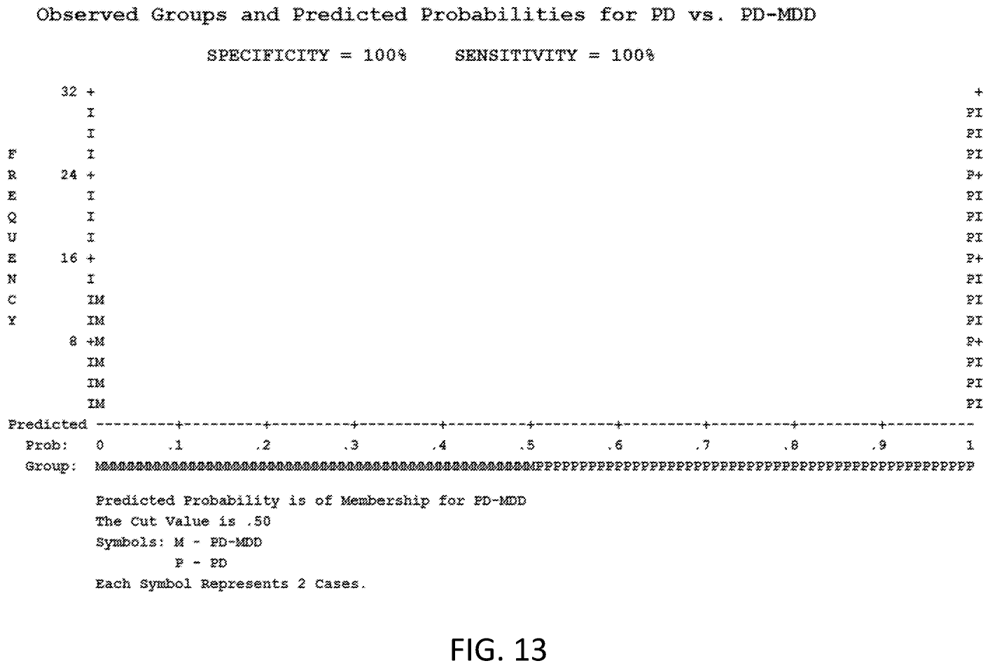

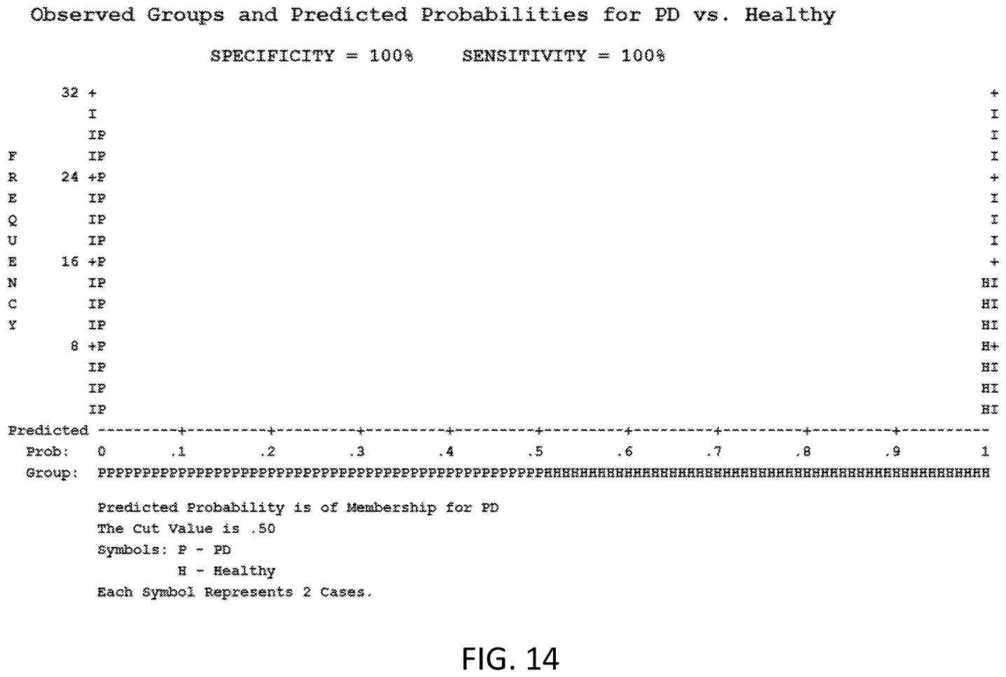

FIG. 13 is a drawing showing a classification graph for tests conducted in connection with the system of the present disclosure to differentiate PD from PD-MDD and FIG. 14 is a drawing showing a classification graph for tests conducted in connection with the system of the present disclosure to differentiate PD from a healthy subject. As can be seen in FIG. 13, a forward binomial logistic regression classification graph shows a predicted probability of membership for PD-MDD where the cutoff value can be 0.50 and each symbol represents two cases. In FIG. 13, M denotes PD-MDD and P denotes PD. As can be seen in FIG. 14, a forward binomial logistic regression classification graph shows a predicted probability of membership for PD where the cutoff value can be 0.50 and each symbol represents two cases. In FIG. 14, P denotes PD and H denotes a healthy subject.

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure.

What is claimed is:

1. A system for evaluating an individual comprising:
a smart device for displaying at least one image associated with a cognitive task and receiving input data from an individual performing the cognitive task;
a remote device including a memory and a processor, the remote device receiving data from the smart device associated with the cognitive task performed by the individual;
the remote device (i) processing the received data by computational analysis to determine learning parameters associated with a performance of the individual, and (ii) evaluating, based on the determined learning parameters and a classification algorithm, the individual to determine whether the individual has a disorder.

2. The system of claim 1, wherein if the individual is determined to have a disorder, the system refers the individual for further evaluation.

3. The system of claim 1, wherein if the individual is determined to have a disorder, the individual is provided with medical treatment.

4. The system of claim 1, wherein the computational analysis includes artificial intelligence trial-by-trial analysis.

5. The system of claim 1, wherein the smart device provides the individual with feedback in response to the received input data through the cognitive task, the feedback being at least one of positive feedback or negative feedback, reversal of feedback, outcome devaluation, and correct feedback or incorrect feedback.

6. The system of claim 1, wherein the cognitive task dynamically changes based on prior responses of the individual.

7. The system of claim 1, wherein the data associated with the cognitive task is analyzed by utilizing trial-by-trial computational models and artificial intelligence approaches to assess parameters for reinforcement learning, gain learning, loss learning, stimulus-by-stimulus response, and drift diffusion.

8. The system of claim 1, wherein the classification algorithm at least one of a positive feedback accuracy, a response time to positive feedback, a negative feedback accuracy, and a response time to negative feedback as a cognitive predictor in evaluating the individual.

9. The system of claim 1, wherein the system utilizes at least one of a positive learning rate, a negative learning rate, a separation threshold, a difference in the speed of response for the execution of responses, and a drift rate for negative feedback as a computational or artificial intelligence predictor in evaluating the individual.

10. A method for evaluating an individual:
displaying at least one image associated with a cognitive task on a smart device;
receiving input data from the individual for performing the cognitive task;
receiving data from the smart device associated with the cognitive task performed by the individual;
processing the received data by computational analysis; and
evaluating, based on the processed data and a classification algorithm, the individual to determine whether the individual has a disorder.

11. The method of claim 10, further comprising determining, by trial-by-trial computational and artificial intelligence analysis, learning parameters according to a performance of the individual.

12. A system for evaluating an individual comprising:
a smart device having a display, the smart device displaying at least one image associated with a cognitive task and receiving input data from an individual for performing the cognitive task; and
a server including a memory and a processor, the server receiving data from the smart device associated with the cognitive task performed by the individual;
the server evaluating, based on the received data and a classification algorithm or an artificial intelligence approach, whether the individual has a disorder.

13. The system of claim 12, wherein if the individual is determined to have a disorder, the system refers the individual for further evaluation.

14. The system of claim 12, wherein if the individual is determined to have a disorder, the individual is provided with medical treatment.

15. The system of claim 12, wherein the smart device (i) processes the received data by computational analysis and artificial intelligence trial-by-trial analysis, to determine learning parameters according to a performance of the individual, and (ii) determines, based on the determined learning parameters and the classification algorithm whether the participant has the disorder.

16. The system of claim 12, wherein the smart device provides the individual with feedback in response to the received input data through the cognitive task, the feedback being at least one of positive feedback or negative feedback, reversal of feedback, outcome devaluation, and correct feedback or incorrect feedback.

17. The system of claim 12, wherein the cognitive task dynamically changes based on prior responses of the individual.

18. The system of claim 12, wherein the data associated with the cognitive task is analyzed by utilizing trial-by-trial computational models and artificial intelligence approaches to assess parameters for reinforcement learning, gain learning, loss learning, stimulus-by-stimulus response, and drift diffusion.

19. The system of claim 12, wherein the classification algorithm utilizes at least one of a positive feedback accuracy, a response time to positive feedback, a negative feedback accuracy, and a response time to negative feedback as a cognitive predictor in evaluating the individual.

20. The system of claim 12, wherein the system utilizes at least one of a positive learning rate, a negative learning rate, a separation threshold, a difference in the speed of response for the execution of responses, and a drift rate for negative feedback as a computational or artificial intelligence predictor in evaluating the individual.
